SEQUENCE LISTING (10 sequences)

SEQ ID NO: 1 - DNA, 750 bp, Mycobacterium bovis
atggggggttctcatcatcatcatcatcatggtatggctagcatgactggtggacagcaaatgggtcggactctgtacgacgatgacgataaggatctcatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcaggatccatggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttcgaattcgaagcttggctgttttggcggatgagagaagattttcagcctgatacagattaa

SEQ ID NO: 2 - protein, 249 aa, Mycobacterium bovis
MGGSHHHHHHGMASMTGGQQMGRTLYDDDDKDLMTEQQWNFAGIEAAASAIQGNVTSIHSLLDEGKQSLTKLAAAWGGSGSEAYQGVQQKWDATATELNNALQNLARTISEAGQAMASTEGNVTGMFAGSMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFEFEAWLFWRMREDFQPDTD

SEQ ID NO: 3 - DNA, 291 bp, Mycobacterium bovis
gatctcatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgca

SEQ ID NO: 4 - protein, 97 aa, Mycobacterium bovis
DLMTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 5 - DNA, 300 bp, Mycobacterium bovis
atggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttc

SEQ ID NO: 6 - protein, 100 aa, Mycobacterium bovis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 7 - DNA, 26 bp, Artificial Sequence
  Description of Artificial SequenceForward Primer for CFP-10
aaggatccatggcagagatgaagacc

SEQ ID NO: 8 - DNA, 29 bp, Artificial Sequence
  Description of Artificial SequenceReverse Primer for CFP-10
aagaagaattcgaagcccatttgcgagga

SEQ ID NO: 9 - protein, 25 aa, Artificial Sequence
  Description of Artificial SequenceSignal Peptide
MAISGVPVLGFFIIAVLMSAQESWA

SEQ ID NO: 10 - protein, 4 aa, Artificial Sequence
  Description of Artificial SequenceSignal Peptide
KDEL

(19) United States
(12) Patent Application Publication
(10) Pub. No.: US 2006/0024332 A1
(43) Pub. Date: Feb. 2, 2006

(54) Recombinant ESAT-6:CFP-10 fusion protein useful for specific diagnosis of tuberculosis

(76) Inventors: Wade R. Waters, Stratford, IA (US); Mitchell V. Palmer, Nevada, IA (US); Frank C. Minion, Ames, IA (US)

Correspondence Address: USDA-ARS-OFFICE OF TECHNOLOGY TRANSFER;NATIONAL CTR FOR AGRICULTURAL UTILIZATION RESEARCH, 1815 N. UNIVERSITY STREET, PEORIA, IL, 61604, US

(21) Appl. No.: 10/909,957

(22) Filed: Aug. 2, 2004

(51) Int. Cl. A61K 39/04; C07K 14/35

(52) U.S. Cl. 424/248.1; 530/350

(57) ABSTRACT

The fusion protein rESAT-6:CFP-10 is useful for differentiating infection of an animal with Mycobacterium bovis from exposure of the animal to other species of Mycobacteria, especially M. avium and M. avium subspecies paratuberculosis (Map). Cells stimulated with the fusion protein are capable of eliciting a variety of in vivo and in vitro responses (e.g. hypersensitivity skin response, IFN-γ, nitric oxide, and TNF-α) indicative of M. bovis infection. This invention will facilitate diagnosis of tuberculosis in cattle, reindeer and other susceptible animal species, thereby preventing unnecessary slaughter of uninfected animals suspected of having of the disease.

### BACKGROUND OF THE INVENTION

1. Field of the Invention

This invention is related to a recombinant ESAT-6:CFP-10 fusion protein and its use for the differentiation of Mycobacterium bovis infection in susceptible animals from infection by M. avium subsp. paratuberculosis, M. avium subsp. avium and many other non-tuberculous Mycobacteria spp.

2. Description of the Prior Art

Mycobacterium bovis, a member of the M. tuberculosis complex, has a wide host range as compared to other species in this disease complex, is infectious to humans, and is the species most often isolated from tuberculous cattle. Control of M. bovis in cattle is particularly difficult due to the presence of wildlife reservoirs such as white-tailed deer, European badgers, and brush-tailed possums. Recently, there has been an increase in the prevalence of M. bovis infection within the United States (US). Detection of tuberculous cattle in Michigan, California, Texas, and New Mexico has resulted in the loss of the TB-free designation for these states (or portions thereof) and subsequent economic losses from increased TB testing costs and hindrances of interstate and international shipment of livestock from these zones. Tuberculosis due to M. bovis in captive Cervidae was identified as an important disease in the US in 1990 and prompted the addition of captive Cervidae to the USDA Uniform Methods and Rules (UM&R) for its eradication. In 1994, a free-ranging white-tailed deer (Odocoileus virginianus) in Michigan was diagnosed with tuberculosis due to M. bovis (Schmitt et al., 1997). Subsequent surveys conducted by the Michigan Department of Natural Resources and Michigan State University Animal Health Diagnostic Laboratory identified a focus of M. bovis infection in free-ranging white-tailed deer in northeast Michigan (Schmitt, S. M., 1997, Journal of Wildlife Diseases, 33:749-758; O'Brien et al., 2001, Journal of Wildlife Diseases, 37:608-613). Tuberculosis in both captive and free-ranging Cervidae represents a serious challenge to eradication of M. bovis infection from the United States. M. bovis infection of reindeer (Rangifer tarandus) is rare, especially in North America where there are no published reports of tuberculosis occurring in reindeer. Despite the low incidence of disease, reindeer are subject to regulations in the USDA Uniform Methods and Rules for the Eradication of Bovine Tuberculosis (TB) APHIS circular #91-45-011 requiring TB testing for interstate movement and herd accreditation.

The demand for improved diagnostic capabilities is again being realized and emphasized with the increased TB testing in the United States. Tests currently approved for the detection of tuberculosis in cattle include measurement of delayed-type hypersensitivity responses (i.e., skin testing) to purified protein derivative (PPD) antigens and an in vitro assay for interferon (IFN)-γ produced in response to PPD stimulation (i.e., Bovigam™, Biocor Animal Health, Omaha, Nebr.). For reindeer, white-tailed deer and other members of the family Cervidae within the US, the single cervical test (SCT, a measure of delayed type hypersensitivity) is the primary approved test for TB. The SCT relies on in vivo reactivity to M. bovis PPD injected intradermally into the mid-cervical region. Deer classified as reactor or suspect with this test are often re-tested using the comparative cervical skin test (CCT) in which M. bovis PPD is injected at one site and M. avium PPD at a separate site. In principle, the CCT provides an added ability to distinguish M. avium responders from M. bovis responders. The in vitro method of TB diagnosis has been developed (Wood P. R. et al., 1994, Vet Microbiol, 40:125-135) and approved for use in cattle in the US as a complimentary test (i.e., in conjunction with the skin test) (Massengill C. E., 2002, Report of the committee on tuberculosis., United States Animal Health Association Annual Meeting, St. Louis, Mo., pp. 590-611). The in vitro assay detects IFN-γ produced by peripheral blood mononuclear cells exposed to no antigen (i.e., background response), M. avium PPD, M. bovis PPD, or mitogen (e.g., pokeweed mitogen, PWM) (Wood P. R. et al., 1991, Aust Vet J, 68:286-290). The assay is particularly amenable for diagnostic laboratories as whole blood cultures are used, thus, circumventing the need for cumbersome cell separation techniques.

A test similar to the Bovigam™ assay designed to detect IFN-γ produced by red deer (Cervus elaphus) leukocytes (i.e., Cervigam™, Biocor Animal Health) also reacts with IFN-γ produced by white-tailed deer (Odocoileus virginianus) leukocytes, indicating that antibodies within the assay are cross reactive with IFN-γ from at least 2 cervid species (Palmer M. V. et al., 2004, J Vet Diagn Invest, 16:16-21; Waters W. R. et al., 2004, J Wildl Dis, 40:66-78). In vitro-based tests such as the Cervigam™ Assay are particularly appealing for use in reindeer and other deer species because animals are handled only once for this test, thereby minimizing capture-associated injuries that are more likely with multiple handling events.

A major limitation of these tests is the cross reactivity of M. bovis PPD (PPDb) with responses induced by exposure to related bacteria, especially M. avium subsp. avium (M. avium) and M. avium subsp. paratuberculosis. Accordingly, the challenge remains to find an agent that can be used to differentially detect M. bovis-infected cattle from cattle infected with M. avium or Map.

Early secretory antigenic target-6 (ESAT-6) and culture filtrate protein-10 (CFP-10) are both IFN-γ-inducing antigens of tuberculous mycobacteria. ESAT-6 and CFP-10 are co-secreted proteins that naturally form a tight 1:1 complex upon export (Renshaw P. S. et al., 2002, J. Biol. Chem., 277:21598-21603). Genes for these proteins are absent in many environmental, non-tuberculous mycobacteria as well as in the TB vaccine strain, M. bovis BCG. Use of ESAT-6 and/or CFP-10 for the stimulating antigen in IFN-γ-based TB assays enhances the specificity when compared to the standard test using PPDb as the stimulating antigen (Buddle B. M. et al., 2003, Vet. Rec., 153:615-620; Buddle B. M. et al., 2001, Vet. Microbiol., 80:37-46; Pollock J. M. et al., 1997, J. Infect. Dis., 175:1251-1254; Pollock J. M. et al., 2000, Vet. Rec., 146:659-665; Vordermeier H. M. et al., 2001, Clin. Diagn. Lab. Immunol., 8:571-578). It has also been shown that T-cell responses to recombinant forms of ESAT-6 and CFP-10 and T-cell responses to mixtures of synthetic overlapping peptides are highly correlated and result in identical sensitivity and specificity, indicating that peptides can be used for detection of TB.

### SUMMARY OF THE INVENTION

We have now devised a novel fusion protein referred to as “rESAT-6:CFP-10” and a method of using it for differentiating infection of an animal with M. bovis from exposure of the animal to other species of non-tuberculous Mycobacteria. The method comprises the steps of:
a. stimulating an animal cell with a fusion protein rESAT-6: CFP-10;
b. measuring a response of the cell to the stimulation in step (a);
c. comparing the response in (b) to a predetermined scale of responses indicative of infection by M. bovis.

The method of the invention is useful in both in vivo and in vitro diagnostic assays including, for example, hypersensitivity skin response, IFN-γ response, nitric oxide response, TNF-α response, and others.

It is an object of this invention to provide a novel means for diagnosing infections by M. bovis in animals.

More particularly, it is also an object of the invention to provide a means for stimulating animal cells so as to enable differentiation of infection by M. bovis from responses induced by exposure of the animal to related bacteria, especially M. avium and M. avium subspecies paratuberculosis.

It is a further object of the invention to facilitate diagnosis of M. bovis in an effort to control tuberculosis in cattle, white-tailed deer and other susceptible animal species.

Another object of the invention is to enable non-comparative diagnostic assays for M. bovis in animals.

Still another object of the invention is to provide an improved tuberculosis test for animals that would avoid unnecessary slaughter of animals falsely identified as reactors.

Other objects and advantages of this invention will become readily apparent from the ensuing description.

### BRIEF DESCRIPTION OF THE FIGURES

FIG. 1 is a map of pISM2202.

FIG. 2 is the sequence of rESAT-6:CFP-10 fusion protein.

FIG. 3 is bar graph depicting delayed-type hypersensitivity responses elicited by PPDs upon experimental infection in cattle. PPDs from M. avium, M. avium subsp. paratuberculosis, and M. bovis were used. Each value is the mean change (in millimeters) in skin thickness±standard error of the mean (error bar) 72 h after injection of PPDs. Skin tests were performed approximately 9 months postchallenge. Values that were statistically significantly different (P<0.05) from the response by control cattle (asterisk) are indicated. The response to PPDb by M. bovis-infected cattle that is statistically significantly different (P<0.05) from concurrent responses to M. avium subsp. paratuberculosis PPD and PPDa is also indicated by the letter “a”.

FIG. 4 is a bar graph depicting IFN-γ responses upon experimental infection in cattle. IFN-γ responses (whole-blood assay) were unaffected (P>0.05) by length of postinfection period. For this reason, each value represents the mean IFN-γ response (in nanograms per milliliter) (i.e., the response to antigen minus the response to medium alone after 48 h) during the period spanning 4 to 7 months postinfection±standard error of the mean (error bar) (n=4). The responses by M. bovis-infected animals that differ significantly, (P<0.05) from the responses by control animals and by M. avium- and M. avium subsp. paratuberculosis (Map)-infected animals for the particular antigen stimulation are indicated (asterisk).

FIG. 5 is a bar graph depicting nitric oxide (NO) responses upon experimental challenge in cattle. NO responses by PBMC were unaffected (P>0.05) by length of postinfection period. For this reason, each value represents the mean NO response (in nanograms per milliliter) (i.e., the response to antigen minus the response to medium alone after 72 h)±standard error of the mean (error bar) during the period spanning 5 to 7 months postinfection (n=3). The response by M. bovis-infected animals that differs significantly (P<0.05) from the responses by control animals and by M. avium- and M. avium subsp. paratuberculosis (Map)-infected animals for the particular antigen stimulation is indicated (asterisk).

FIG. 6 is a line graph showing longitudinal IFN-γ response to rESAT-6:CFP-10 fusion protein in cattle. Values indicate the mean responses to rESAT-6:CFP-10 stimulation (48 h) (in nanograms per milliliter) minus the response to medium alone standard error of the mean (error bars). Responses by M. bovis-infected animals that were significantly different (P<0.05) from the responses by control animals and by M. avium- and M. avium subsp. paratuberculosis (Map)-infected animals are indicated (**). The response by M. bovis-infected animals that was significantly different from the responses by control animals (P<0.05), M. avium-infected animals (P=0.08), and M. avium subsp. paratuberculosis-infected animals (P<0.05) is indicated

FIG. 7 is a bar graph showing mean IFN-γ responses in experimentally injected reindeer to recombinant ESAT-6, CFP-10, and ESAT6:CFP10. Treatments included no stimulation (PBS), rESAT-6 (10 μg/ml), rCFP-10 (10 μg/ml), or rESAT-6:CFP-10 (10 μg/ml). Data are presented as the response to antigen stimulation minus the response to no stimulation (ΔIFN-γ, OD), mean±SEM. Responses at 90d, 103d, 120d and 160d after challenge were averaged (n=4) with open bars representing responses by M. bovis-infected reindeer (n=10) and closed bars representing responses by non-infected reindeer (n=4). Asterisks indicate responses by M. bovis-infected reindeer differ (P<0.05) from respective responses by non-infected reindeer.

FIG. 8 depicts longitudinal responses by M. bovis-infected reindeer (n=10) to recombinant proteins. The symbol “a” indicates responses to rESAT-6 differ (P<0.05) from respective responses to rCFP-10 and rESAT-6:CFP-10.

FIG. 9 depicts effects of injection of purified protein derivatives (PPD's) for skin test on IFN-γ responses to mycobacterial antigens and mitogen in reindeer. Reindeer were tested for delayed type hypersensitive responses to mycobacterial antigens at 240 d after challenge with M. bovis. Heparinized blood samples were obtained from reindeer at indicated time points and dispensed in 1.5 ml aliquots immediately into individual wells of a 24-well plate. Treatments included no stimulation (PBS), M. avium PPD (40 μg/ml), M. bovis PPD (40 μg/ml), rESAT-6:CFP-10 (10 μg/ml), or pokeweed mitogen (PWM, 20 μg/ml). Data are presented as the response to antigen stimulation minus the response to no stimulation (ΔIFN-γ, OD), mean±SEM.

### Deposit of Biological Material

Esherichia coli containing plasmid pISM1543 was deposited on Jul. 23, 2004, under the provisions of the Budapest Treaty in the Agricultural Research Culture Collection (NRRL) in Peoria, Ill., and has been assigned Accession No. B-30756.

### DETAILED DESCRIPTION

Source Material and Definitions.

The fusion protein of this invention comprises two immune enhancing (IFN-γ-inducing) antigens, ESAT-6 (95 residues) and CFP-10 (100 residues). Though genes for these proteins, esat-6 and cfp-10, respectively, are known to be absent in many environmental, non-tuberculous mycobacteria as well as in the TB vaccine strain, M. bovis BCG, they are readily available from several common mycobacteria known to excrete the ESAT-6 and CFP-10 proteins, including for example, field strains (wild-types) of M. tuberculosis, M. bovis, M. kansasii, M. marinum, M. leprae, and M. smegmatis. It is contemplated that esat-6 and cfp-10 genes from any single species of mycobacterium or from any combination of mycobacteria could be used in the making of an expression system for the rESAT-6:CFP-10 fusion protein of the invention.

Also encompassed herein are DNA sequences encoding the aforementioned rESAT-6:CFP-10 fusion protein as well as vectors containing the DNA. Any such vector may contain a transcriptional regulatory element (TRE) operably linked to the DNA. Cells containing the expression vectors of the invention are also encompassed. As used herein, “operably linked” means incorporated into a genetic construct so that expression control sequences effectively control expression of a coding sequence of interest.

In another aspect, the invention encompasses a method of inducing an in vitro immune response to an immunogenic molecule comprising culturing the fusion protein of the invention with a T-cell and an antigen presenting cell (APC). The invention also features a method of inducing an in vivo immune response to an immunogenic molecule involving delivering the fusion protein of the invention to an immune system of a subject animal. Exemplary of an in vivo response would be a delayed type hypersensitivity response, as manifest for example in a skin test. Of specific interest is inducing an in vivo or an in vitro immune response that can be used to quantitatively differentiate infection by M. bovis from incidental exposure of the host, or cells from the host, to non-tuberculous mycobacteria.

The terms “polypeptide” and “protein” are used interchangeably herein, and mean any peptide-linked chain of amino acids, regardless of length or post-translational modification.

As used herein, an “immune response stimulating effect” and “immune response enhancing effect” are used to refer to the immunological effect that the subject fusion protein has on an animal, particularly a mammalian animal, or on a cell in culture, particularly a mammalian cell, when the fusion protein is administered to a mammalian subject or when said cell is subjected to the fusion protein when that response is significantly greater than the response that would occur in the absence of the fusion protein. The effect may include, without limitation, induction of cytokine secretion (e.g. IFN-γ, TFN-α) and the like.

Fusion Proteins.

In the preferred embodiment of the invention, the rESAT-6:CFP-10 fusion protein comprises the complete amino sequence of each of the constituent polypeptides. That is, the amino acid sequence of each domain of the fusion protein can be identical to the wild-type sequence of the corresponding polypeptide. However, there is basis in the literature for employing variations of the complete sequences. As used herein, a variation is defined as comprising substantial equivalents of either or both of the ESAT-6 and CFP-10 antigens, including immunological fragments thereof, and including proteins having deletions, additions, and substitutions in either or both of the ESAT-6 and CFP-10. Any variation must be effective to stimulate a T-cell response to Mycobacterium bovis that is significantly greater than a T-cell response to a non-tuberculous mycobacterium in the absence of stimulation by the fusion protein. The expression “immunologically equivalent” as used herein in reference, for example, to a fragment of ESAT-6, CFP-10, or the rESAT-6:CFP-10 fusion protein is intended to mean that the immunological response stimulated by that fragment is substantially equivalent to the response stimulated by the native antigen as measured by standard diagnostic assays. Examples of suitable variations and basis for their functionality are described below. As previously mentioned, ESAT-6 is a potent inducer of IFN-γ. It has been shown that ESAT-6 comprises multiple subregions that also have potential for IFN-γ induction. For example, synthetic peptides containing amino acids 1 to 20 and amino acids 10 to 30 of ESAT-6 were presented by various human leukocyte anitgen (HLA)-DR molecules and thereby elicited IFN-γ production by activated CD4+ T cells (Ulrichs et al., 1998, Eur. J. Immunol., 28:3949-3958). It has also been shown that T-cell responses to recombinant ESAT-6 (rESAT-6) and recombinant CFP-10 (rCFP-10) and those to mixtures of synthetic overlapping peptides spanning the complete amino acid sequence of each antigen are highly correlated and result in identical sensitivity and specificity (Arend S. M. et al., 2000, Infection and Immunity, 68:3314-3321). Accordingly, each domain (immune enhancing molecule) of the fusion protein can contain deletions, additions, or substitutions in the native ESAT-6 and/or CFP-10 sequences. Contemplated herein are immune enhancing molecules having at least 50% (e.g., 50%, 60%, 70%, 80%, 90%, 95%, 99%, 100% or even more) of the ability of each wild-type polypeptide in the full length rESAT-6:CFP-10 to stimulate a Th1 type immune response. Also contemplated herein are immune enhancing molecules comprising domains having at least 80%, at least 90%, at least 95%, at least 98%, and at least 99% sequence identity to the respective ESAT-6 and CFP-10 domains of the native M. bovis peptides represented by SEQ ID NOs:5 and 6. Methods of comparing the relative ability of two or more molecules to enhance a particular immune response are known in the art. Substitutions will preferably be conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. Also contemplated herein are artificial immune enhancing proteins that contain whole or subregions of one or both ESAT-6 and CFP-10. The relevant subregions would be those with the ability to enhance an immunogenic response.

The rESAT-6:CFP-10 fusion protein of the invention may be produced substantially as described in Example 1, below, or by any alternative means within the skill of a person in the art. The ESAT-6 and CFP-10 can be disposed in any convenient orientation with respect to each other in the fusion proteins of the invention. Thus, for example, the ESAT-6 can be N-terminal of the CFP-10, or vice versa. The two peptides can be immediately adjacent to each or they can be separated by a linker peptide and/or restriction site. Linker peptides can be 1 to about 30, even 50, amino acids long and can contain any amino acids. In general, a relatively large proportion (e.g., 20%, 40%, 60%, 80%, 90%, or 100%) of the amino acid residues in the linker will be glycine and/or serine residues. Such linkers can contain, for example, one or more (e.g., two, three, four, five, six, seven, eight, nine, ten, or more) gly-gly-gly-ser (GGGS) units. Linking of one non-protein molecule to another or of one non-protein molecule to a protein molecule can be achieved by standard chemical methods known in the art. Any restriction enzymes such as BamHI or EcoRI used in the construct described in Example 1 may be incorporated into the fusion protein.

Smaller fusion proteins (less than 100 amino acids long) can be conveniently synthesized by standard chemical means. In addition, the fusion proteins can be produced by standard in vitro recombinant DNA techniques and in vivo recombination/genetic recombination (e.g., transgenesis), using the nucleotide sequences encoding the appropriate polypeptides or peptides. The fusion proteins can also be made by a combination of chemical and recombinant methods.

The fusion proteins of the invention also include those described above, but which contain additional amino acid segments. Thus the fusion proteins can contain, for example, a hydrophobic signal peptide. The signal peptide is generally immediately N-terminal of the mature polypeptide (fusion protein) but can be separated from it by one or more (e.g., 2, 3, 4, 6, 8, 10, 15 or 20) amino acids, provided that the leader sequence is in frame with the nucleic acid sequence encoding the fusion protein. The signal peptide, which is generally cleaved from proteins prior to secretion, directs proteins into the lumen of an appropriate cell's endoplasmic reticulum (ER) during translation and the proteins are then secreted, via secretory vesicles, into the environment of the cell. Useful signal peptides can be the native signal peptide of the relevant immune enhancing molecule or a functional fragment of the native signal peptide, i.e., a fragment of the signal peptide that substantially the same signal activity as the full-length leader. Alternatively, the signal peptide can be that of another exported polypeptide. For example, the signal peptide can have the amino acid sequence MAISGVPVLGFFIIAVLMSAQESWA (SEQ ID NO:9). In addition, the peptide sequence KDEL (SEQ ID NO:10) has been shown to act as a retention signal for the ER.

The fusion proteins of the invention can also be modified for in vivo use by the addition, at the amino- and/or carboxyl-terminal ends, of a blocking agent to facilitate survival of the relevant polypeptide in vivo. This can be useful in those situations in which the polypeptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to the person in the art. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety.

Nucleic Acids Encoding Fusion Proteins.

The invention includes nucleic acids (e.g., cDNA, genomic DNA, synthetic DNA, or RNA) encoding the rESAT-6:CFP-10 fusion proteins. The nucleic acids can be double-stranded or single-stranded (i.e., a sense or an antisense strand). A RNA molecule can be produced by in vitro transcription. The nucleic acid molecules are not limited to coding sequences and can include some or all of the non-coding sequences that lie upstream or downstream of a particular coding sequence. The nucleic acids can have nucleotide sequences that are identical to those of nucleic acids encoding the wild-type immune enhancing molecules. Alternatively, they can contain codons other than wild-type codons but which, due to the degeneracy of the genetic code, encode immune enhancing polypeptides with amino acid sequences identical to relevant wild-type polypeptides. Furthermore, the nucleic acids can encode immune polypeptides (or functional fragments thereof) with any of the above described deletions, additions, or substitutions. In addition, the nucleic acids can contain nucleotide sequences encoding functional fragments of immune enhancing polypeptides.

The nucleic acids of the invention are essentially “hybrid genes,” containing at least two portions. One portion encodes the ESAT-6 polypeptide and another portion encodes the CFP-10 polypeptide. Where the fusion protein encoded by the nucleic acid contains more than two domains, the appropriate nucleic acid will contain a corresponding number of portions. Between the first and second portions (and any additional portions) can be codons encoding a linker (see above).

Expression Vectors and Systems.

The invention also contemplates vectors, particularly expression vectors, useful for the expression of a nucleic acid sequence encoding a fusion protein of interest with an initiator methionine and, preferably, a signal sequence. The aforementioned nucleic acid is operably linked to one or more transcriptional regulatory elements (TRE), e.g., a promoter or enhancer-promoter combination, as known in the art. A list of promoters is provided in Table 1 of U.S. Pat. No. 6,537,552 B1, herein incorporated by reference.

Methods well known to those skilled in the art can be used to construct expression vectors containing relevant coding sequences and appropriate transcriptional/translational control signals. See, for example, the techniques described in Sambrook et al., Molecular Cloning: A Laboratory Manual (2nd Ed.) [Cold Spring Harbor Laboratory, N.Y., 1989], and Ausubel et al., Current Protocols in Molecular Biology, [Green Publishing Associates and Wiley Interscience, N.Y., 1989]. Expression systems that may be used for small or large scale production of the fusion proteins include, but are not limited to, microorganisms such as bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecules of the invention (see below); insect cell systems infected with recombinant virus expression vectors (for example, baculovirus) containing the nucleic acid molecules of the invention; plant cell systems infected with recombinant virus expression vectors [for example, cauliflower mosaic virus (CaMV) and tobacco mosaic virus (TMV)] or transformed with recombinant plasmid expression vectors (for example, Ti plasmid) containing fusion protein nucleotide sequences; or mammalian cell systems (for example, COS, CHO, BHK, 293, VERO, HeLa, MDCK, W138, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal, transfected with a plasmid vector or infected with a viral vector.

Basis for the Invention.

The basis for the invention is the discovery that responses to rESAT-6:CFP-10 were not induced upon experimental inoculation of calves with doses of M. avium or M. avium subsp. paratuberculosis that clearly sensitized these animals to the respective mycobacterium. In contrast, M. bovis-infected calves exhibited responses to rESAT-6:CFP-10 equal to those induced by stimulation with PPDb. Therefore, even under conditions of high exposure to M. avium- or M. avium subsp. paratuberculosis rESAT-6:CFP-10 is specific for diagnosis of bovine tuberculosis. By virtue of CFP-10 being highly soluble in comparison with ESAT-6, solubility of the ESAT-6 domain in the fusion protein is enhanced, thereby rendering the fusion protein more stable and immunologically relevant in comparison to the ESAT-6 alone.

Stimulating an Immune Response In Vivo for M. bovis Detection.

In practice, animals to be tested are injected with the fusion protein and a response is subsequently observed. The amount and site of the fusion protein injection, and the delay period for observing the response are, of course, dependent upon the size and species of animal, and other parameters related to the protocol for the specific diagnostic assay being applied. In the case of a delayed-type hypersensitivity response (skin test) in cattle, for example, the animal is injected 10-10001 μl (0.1-10 mg/ml), preferably about 100 μl (1 mg/ml) of the fusion protein. The injection site is typically in the cervical region or tail base but could be at other locations as well. Dermal reactions may be observed visually in terms of welt thickness and/or diameter. The reaction sites may also be biopsied and stained with hematoxylin and eosin (H/E) to determine the extent of the response.

Stimulating an Immune Response In Vitro for M. bovis Detection.

Cells isolated from an animal suspected of potential M. bovis infection may be cultured in the presence of the ESAT-6:CPF-10 fusion protein. For example, peripheral blood mononuclear cells (PBMC) isolated from buffy coat fractions of peripheral blood can be cultured in microtiter plates with a suitable medium in the presence of the rESAT-6:CFP-10. Culture supernatants are then harvested and stored for subsequent analysis.

A typical assay for M. bovis infection in the culture supernatants would detect the level of IFN-γ production such as by ELISA as described in Example 1. Nitric oxide (NO) responses to the fusion protein is also indicative of M. bovis-infected animals. The amount of nitrite, the stable oxidation product within culture supernatants is a correlate of the amount of NO produced by cells in culture. Nitrite can be measured using the Griess reaction, described in further detail in Example 1, below. Likewise, M. bovis infection is indicated by tumor necrosis factor alpha (TNF-α) responses to the fusion protein. TNF-α can be measured using a capture-ELISA, as described further in Example 1, below.

Regardless of the assay format, the advantages of the invention are realized by the ability to conduct a non-comparative test. That is, once the expected level of response to the ESAT-6:CPF-10 fusion protein in M. bovis-infected animals is established and it is shown that the level of response is significantly different from what would be observed in animals that were either non-infected or were incidentally exposed to other mycobacteria, then responders could be readily identified as being M. bovis-infected. Conversely, non-responders could be assumed to not be M. bovis-infected. Responders would be those animals that yielded a response above some predetermined threshold for the particular assay being applied, and non-responders would be those animals yielding a response below that threshold. It would be within the skill of a person in the art to experimentally establish the threshold for a given assay by running a controlled experiment with non-infected animals and/or animals that have been experimentally infected with the non-tuberculous mycobacteria.

The invention would have utility in diagnostic assays for M. bovis in any animal species susceptible to infection by this organism. M. bovis-tuberculosis is of particular concern in humans and other mammals, particularly in the families Bovidae and Cervidae. The invention would find most widespread application in testing for tuberculosis in meat animals of the Bovidae family, especially cattle, bison, goats, sheep and the like. There would also be application for testing of susceptible species that are traditionally free-ranging, but are subject to wildlife management practices. Examples of such species are reindeer, deer, antelope, elk, etc., and also exotic animals that are maintained in zoos and wildlife parks.

The following examples are intended to further illustrate the invention, without any intent for the invention to be limited to the specific embodiments described therein.

### EXAMPLE 1

### Cattle Study

Animals, Challenge Procedures, and Bacterial Culture.

Cattle were obtained from tuberculosis-free herds. The study consisted of two experiments. In the first experiment (Example 1A), groups consisted of Hereford cattle from a herd with a history of widespread exposure to M. avium (n=8) and M. bovis-challenged crossbred beef cattle (n=8). Calves in this portion of the study were 10 months of age at the time of sample collection. Sixty percent (30 out of 50) of adult cows and 78 percent (32 out of 41) of pre-weaned calves in the herd of origin of the M. avium-exposed calves had positive IFN-γ responses to M. avium PPD [i.e., M. avium PPD (PPDa) stimulation minus no stimulation≧0.05 optical density (OD) units; Bovigam™, Biocor Animal Health]. Calves in the M. bovis-challenged group were tested and confirmed negative for M. bovis and M. avium exposure prior to challenge (Bovigam™, Biocor Animal Health). The M. avium-exposed cattle were housed outdoors whereas M. bovis-infected cattle were housed within a biosafety level-3 (BL-3) confinement facility. For M. bovis infection, the challenge inoculum [2.4×10⁶ colony forming units (cfu) in 0.2 ml of phosphate buffered saline, 0.15 M, pH 7.2 (PBS)] was instilled directly into the tonsillar crypts of anesthetized cattle as previously described for inoculation of white-tailed deer (Palmer M. V. et al., 2004, J Vet Diagn Invest, 16:16-21). The strain of M. bovis used for the challenge inoculum (95-1315, USDA, Animal Plant and Health Inspection Service designation) was originally isolated from a white-tailed deer in Michigan, US (Schmitt S. M. et al., 1997, J. Wildl. Dis., 33:749-58). Inoculum consisted of mid-log-phase M. bovis grown in Middlebrook's 7H9 media supplemented with 10% oleic acid-albumin-dextrose complex (OADC, Difco, Detroit, Mich.) plus 0.05% Tween 80 (Sigma Chemical Co., St. Louis, Mo.). Lesions typical of M. bovis infection were detected in M. bovis-inoculated animals and infection was confirmed by isolation of M. bovis from tissues.

In the second experiment (Example 1B), groups consisted of non-challenged calves (n=4), M. avium-challenged calves (n=4), M. avium subsp. paratuberculosis (hereafter, referred to as “Map”)-challenged calves (n=3), and M. bovis-challenged calves (n=4). Calves (males, castrated at ˜3 months of age) were obtained from tuberculosis- and Map-free Holstein herds at 2-4 days of age and housed indoors in temperature-controlled rooms (18-24° C.; BL-3 facility for M. bovis-infected calves). M. bovis (strain 95-1315) and M. avium (strain 3988, bovine isolate) for challenge inoculum were grown in Middlebrook's 7H9 media (NADC, Ames, Iowa) supplemented with 10% oleic acid-albumin-dextrose complex plus 0.05% Tween 80 (Sigma Chemical Co., St. Louis, Mo.). Medium for Map (strain K10, cattle isolate) was additionally supplemented with 2 mg/L mycobactin J (Allied Monitor Inc., Fayette, Mo.). Challenge inoculum [˜10⁴ cfu M. bovis, ˜10¹⁰ cfu M. avium or ˜10⁸ cfu Map] was instilled directly into tonsillar crypts of sedated calves as described (Waters W. R. et al., 2003, Infect. Immun., 71:5130-5138). Calves in Map- and M. avium-challenged groups received 4 weekly doses of inoculum for a total dose of ˜4×10¹⁰ cfu of M. avium or ˜4×10⁸ cfu of Map.

Cell Culture and Antigens.

Peripheral blood mononuclear cells (PBMC) were isolated from buffy coat fractions of peripheral blood collected in 2× acid citrate dextrose (Burton J. L. et al., 1996, J. Leukocyte Biol., 59:90-99). Individual wells of 96-well round-bottom microtiter plates (Falcon, Becton-Dickinson; Lincoln Park, N.J.) were seeded with 2×10⁵ PBMC in a total volume of 200 μl per well. Medium was RPMI 1640 (GIBCO, Grand Island, N.Y.) supplemented with 2 mM L-glutamine, 25 mM HEPES buffer, 100 units/ml penicillin, 0.1 mg/ml streptomycin, 1% non-essential amino acids (Sigma), 2% essential amino acids (Sigma), 1% sodium pyruvate (Sigma), 50 μM 2-mercaptoethanol (Sigma), and 10% (v/v) fetal bovine sera (FBS). Wells contained medium plus 5 μg/ml PPDb (CSL limited), 5 μg/ml PPDa (CSL Limited), 20 μg/ml M. bovis strain 95-1315 whole cell sonicate (WCS), 20 μg/ml M. avium strain 3988 WCS, 20 μg/ml Map strain K10 WCS, 10 μg/ml rESAT-6:CFP-10, or medium alone (no stimulation). Antigens consisting of sonicates of the challenge strains were prepared as described (Waters W. R. et al., 2003, Clin. Diag. Lab. Immunol., 10:960-966). After incubation of PBMC cultures for 48 h at 37° C. in 5% CO₂, culture supernatants were harvested and stored at −80° C. until thawed for analysis.

Cloning and Expression of rESAT-6:CFP-10 Fusion Protein.

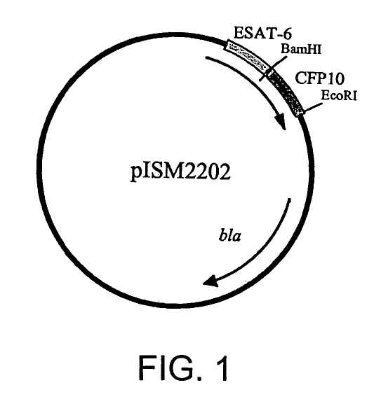

CFP10 was amplified from M. bovis genomic DNA with primers CFP10F (5′-AAGGATCCATGGCAGAGATGAAGACC) (SEQ ID NO:7) and CFP10R (5′-AAGAAGAATTCGAAGCCCATTTGCGAGGA) (SEQ ID NO:8) to incorporate BamHI and EcoRI restriction sites. The PCR products were ligated into pCR2.1 (Invitrogen, Carlsbad, Calif.) and transformed into TOP10F′ chemically competent cells (mcrA Δ(mcrCB-hsdSMR-mrr) (Φ80 lacZ M15) Δ(lac)X74 deoR recA1 araD139 Δ(ara-leu)7697 galU galK rpsL endA1 nupG F′ [(Tet^{r})]). Plasmid DNA was isolated using Qiaspin miniprep system (Qiagen, Valencia, Calif.) and screened by DNA sequencing. The appropriate plasmid was designated pISM2204. The expression vector pISM404 (pTrcHisB:ESAT-6) (Menon S. A. et al., 2002, J. Interferon Cytokine Res., 22:807-813) and pISM2204 were digested with BamHI and EcoRI. The digested DNA was gel purified, ligated, and transformed into DH5αcells [supE44 ΔlacU169 (Φ80 lacZΔM15) hsdR17 recA1 endA1 gyrA96 thi-1 relA1]. Recombinant plasmids were sequenced to confirm the insertion, and the final plasmid was designated pISM2202 (FIG. 1). The origin of both the ESAT-6 and the CFP-10 in the resultant fusion protein is M. bovis strain 95-1315.

rESAT-6:CFP10 protein was purified from a 200 ml 2×YT ampicillin (50 μg/ml) culture, induced at an OD600 of 0.6 with 1 mM isopropyl thiogalactoside for 4 h at 37° C. Cells were lysed in 20 ml of lysis buffer (20 mM Tris pH 8.0, 100 mM NaCl, 8M Urea) followed by sonication. Proteins were purified on 10 ml Ni-NTA resin (Sigma) using the Biologic HR Chromatography System (Bio-Rad, Hercules, Calif.). The resin was washed with 10 ml water followed by 25 ml lysis buffer, and then the cell lysate was added. Nonspecific proteins were washed through the column with 60 ml of lysis buffer followed by 30 ml lysis buffer containing 10 mM imidazole. The protein was eluted with 30 ml of lysis buffer containing 200 mM imidazole. Fractions were collected every 5 ml and analyzed by western blotting against the 6×His Tag (Clontech, Palo Alto, Calif.) (10). Fractions were then dialyzed overnight at 4° C. in PBS and quantified using the Bradford assay.

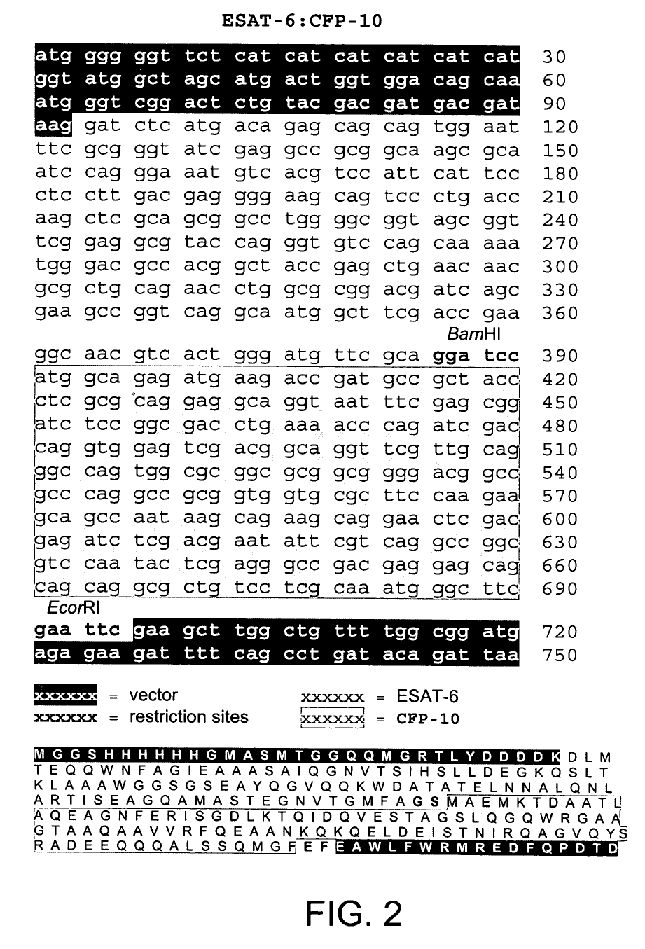

FIG. 2 gives the nucleic acid (SEQ ID NO:1) and the amino acid (SEQ ID NO:2) sequences of the rESAT-6:CFP10 fusion protein, including restriction sites and flanking vector sequences. The nucleic acid and amino acid sequences for ESAT-6, per se, are represented by SEQ ID NO:3 and SEQ ID NO:4, respectively. The nucleic acid and amino acid sequences for CFP-10, per se, are represented by SEQ ID NO:5 and SEQ ID NO:6, respectively.

IFN-γ assay. Heparinized blood samples were dispensed in 1.5 ml aliquots into individual wells of a 24-well plate (Falcon 353047, Becton Dickinson and Company, Franklin Lakes, N.J.). Blood cultures were incubated for 48 h, plasma harvested and stored at −80° C. IFN-γ concentrations in stimulated plasma (i.e., whole blood assay) or supernatants were determined using a commercial ELISA-based kit (Bovigam™, Biocor Animal Health). Absorbencies of standards and test samples were read at 450 nm using an ELISA plate reader (Molecular Devices, Menlo Park, Calif.). In addition, IFN-γ concentrations in supernatants (3 pooled replicate samples) from PBMC cultures were analyzed. Concentrations (ng/ml) in test samples were quantified by comparing absorbencies of test samples with absorbencies of standards within a linear curve fit. Duplicate samples for individual treatments were analyzed.

Griess reaction assay. The amount of nitrite, the stable oxidation product of nitric oxide (NO), within culture supernatants is a correlate of the amount of NO produced by cells in culture. Nitrite was measured using the Griess reaction (Rajaraman V. et al., 1998, J. Dairy Sci., 81:3278-3285) performed in 96-well microtiter plates (Immunolon 2, Dynatech Laboratories, Inc., Chantilly, Va.). Culture supernatant (100 μl) was mixed with 100 μl of Griess reagent (0.5% sulfanilamide, Sigma) in 2.5% phosphoric acid (Mallinckrodt Chemicals, Inc., Paris, Ky.) and 0.05% N-(1-naphthyl) ethylenediamine dihydrochloride (Sigma). The mixture was incubated at 21° C. for 10 min. Absorbencies of test and standard samples at 550 nm were measured using an automated ELISA plate reader. Samples were diluted in culture medium (RPMI 1640 medium with 2 mM L-glutamine and 10% FBS v/v). Absorbencies of standards and test samples were converted to ng/ml of nitrite by comparing them to absorbencies of sodium nitrite (Fisher Chemicals, Fair Lawn, N.J.) standards within a linear curve fit. Assays were run on 3 sets of pooled triplicates for each treatment.

TNF-α Assay.

TNF-α was measured using a capture-ELISA (protocol and reagents provided by L. Babiuk, Veterinary Infectious Diseases Organization, Saskatoon, Saskatchewan, Canada). Assays were performed in Immunolon II microtiter plates (Dynatech Laboratories, Inc., Chantilly, Va.). Reagents consisted of a capture antibody (mouse ascites anti-bovine-TNF-α, IgG fraction), detection antibody (rabbit anti-bovine-TNF-α, IgG fraction), rBoTNF, biotinylated goat anti-rabbit IgG (Zymed Laboratories, Inc., South San Francisco, Calif.), horseradish peroxidase-conjugated streptavidin-biotinylated complex (Amersham Corporation, Arlington Heights, Ill.), substrate (H₂O₂ at 0.1% v/v) and dye (2,2′-azinodi-ethylbenzothiazoline-sulphonic acid). Internal standards consisted of rBoTNF-α diluted serially in PBS (pH 7.2, 0.01 M) with Tween 80 (0.1% v/v) and gelatin (0.5% v/v, PBST-g). Positive and negative controls and test samples also were diluted serially in PBST-g. Capture antibody was diluted (1:1000 v/v) in carbonate buffer (pH 9.6, 0.01M), and detection antibody in PBST-g (1:1500 v/v). Biotinylated goat anti-rabbit Ig was diluted 1:10,000 and horseradish peroxidase-conjugated streptavidin-biotinylated complex 1:2000 in PBST without gelatin. Intervening washes were done with PBST without gelatin. Enzyme substrate and indicator dye were diluted in citrate buffer. Incubations were at room temperature with the exception of capture antibody in carbonate buffer, which was incubated at 4° C. Absorbencies of standards and test samples was read at 405 and 490 nm using an ELISA plate-washer and reader (Dynatech MR7000). TNF-α concentrations (ng/ml) in test samples were determined by comparing the absorbencies of test samples with the absorbencies of standards within a linear curve fit.

Delayed Type Hypersensitivity Responses (i.e., Skin Testing).

Nine months after challenge, calves were tested for in vivo responsiveness to mycobacterial antigens using a modified, comparative cervical skin test (Waters W. R. et al., 2003, Vaccine, 21:1518-1526). Animals were injected intradermally with 100 microliters of PPDb (1 mg/ml), PPDa (0.4 mg/ml) and Map PPD #0202 (1 mg/ml) (National Veterinary Services Laboratory, Ames, Iowa). Data are presented as skin thickness (mm) at 72 h after injection of PPD minus pre-injection skin thickness.

Statistics.

Data were assessed for normality prior to statistical analysis. Arithmetic and log₁₀-transformed data were analyzed as a split-plot with repeated measures ANOVA using Statview software (version 5.0, SAS Institute, Inc., Cary, N.C.). The statistical model included effects of treatments (infection-type and recall antigen), time (months relative to establishment of infection), and the interaction of treatment and time on IFN-γ, TNF-α and NO production by whole blood or enriched PBMC cultures. Scheffe's test was applied when effects (P<0.05) detected by the model were significant (P<0.05).

### Results

Responses to rESAT-6:CFP-10 by Cattle Naturally-Sensitized to M. avium.

Natural exposure of cattle to M. avium was determined by detection of recall IFN-γ responses to PPDa. Seven of 8 calves exposed to M. avium had positive IFN-γ responses to PPDa (i.e., PPDa stimulation minus no stimulation≧0.05 OD). IFN-γ responses to PPDa may also be indicative of Map infection; however, fecal samples from cattle exposed to M. avium and adult members of their herd were negative for Map growth using standard Map culture techniques. IFN-γ responses by PBMC from M. avium-exposed calves to PPDa (7.5 ng/ml±3.0 ng/ml) exceeded (P<0.01) parallel responses to PPDb (−2.4 ng/ml±1.1) (Table 1). Responses of M. bovis-infected calves to PPDa exceeded (P<0.05) those of M. avium-exposed calves, indicating vigorous responses induced by M. bovis infection and the cross reactivity of PPD's. As expected, PPDb-induced IFN-γ production by M. bovis-infected calves (55.2 ng/ml±9.7) exceeded (P<0.01) responses elicited by PPDa (17.3 ng/ml±3.0). IFN-γ responses of M. bovis-infected calves to rESAT-6:CFP-10 also exceeded (P<0.01) those of calves exposed to M. avium. Most importantly, stimulation of PBMC from M. avium-exposed calves with rESAT-6:CFP-10 did not elicit a significant IFN-γ response (Table 1). TNF-α and NO responses to PPDa, PPDb, and rESAT-6:CFP-10 were qualitatively similar to IFN-γ responses (Tables 2 and 3, respectively).

Sensitization Upon Intratonsillar Challenge.

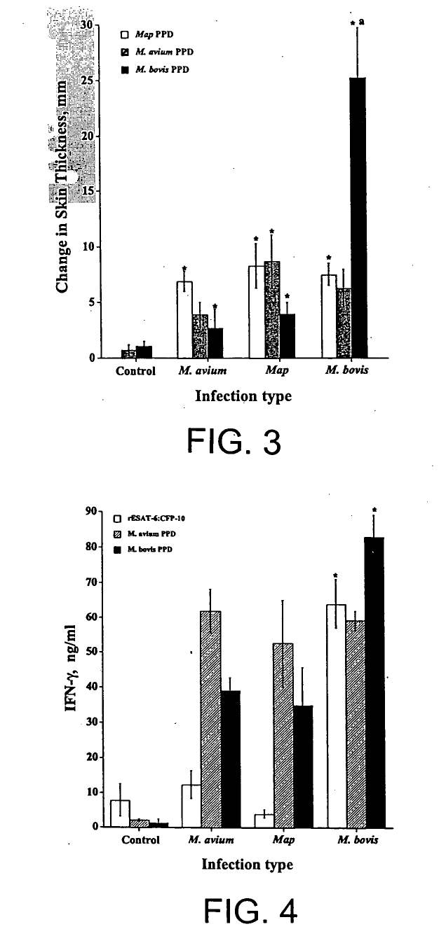

Because the dose and duration of natural exposure to M. avium are not amenable to determination or control, an experimental model of M. avium infection was applied for comparison to responses induced by experimental M. bovis infection (Waters, W. R. et al., 2003, Infect. Immun., 71:5130-5138). At 9 months postchallenge, cutaneous delayed-type hypersensitivity responses to respective PPD's were detected (FIG. 3). With the exception of PPDb-induced responses by M. bovis-infected calves, responses to the various PPD's were similar, indicating cross-reactivity. Recall IFN-γ and NO responses by M. bovis-infected calves to M. bovis WCS exceeded (P<0.05) concurrent responses to M. avium and Map WCSs as well as responses induced by experimental M. avium and Map infection to M. bovis WCS (Tables 4 and 5, respectively). Likewise, IFN-γ and NO responses by M. avium-infected calves to M. avium WCS exceeded (P<0.05) concurrent responses to M. bovis WCS and Map WCS as well as responses by non-infected calves to M. avium WCS (Tables 4 and 5, respectively). Responses by M. avium- and Map-infected calves to M. avium and Map WCSs did not differ reflecting the many conserved antigens of these two closely related subspecies. Analysis of IFN-γ levels in PBMC supernatants and whole blood stimulated cultures revealed similar effects of antigen stimulation and infection status (data not shown).

Differential Diagnosis Using rESAT-6:CFP-10.

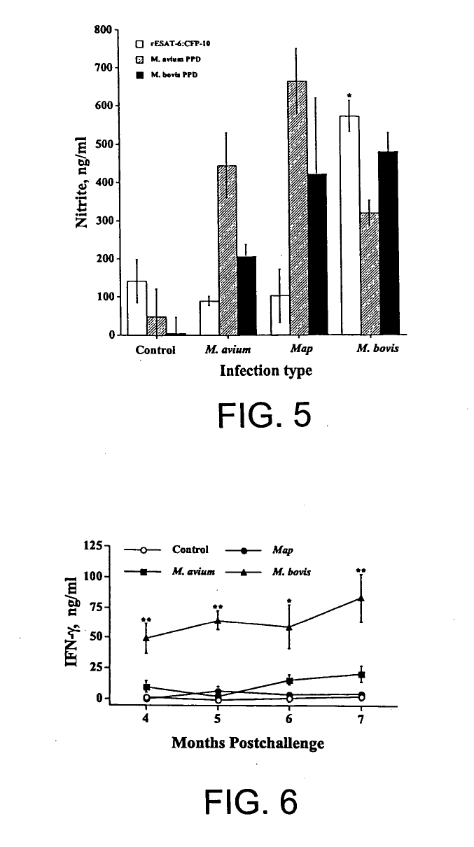

Differential IFN-γ responses to PPDa and PPDb are currently used for M. bovis diagnosis (Bovigam™, Biocor Animal Health). For this reason, responses to rESAT-6:CFP-10 were compared with responses to PPDa and PPDb. IFN-γ responses of M. bovis-infected calves to PPDb exceeded (P<0.05) that of noninfected, M. avium-, and Map-infected calves (FIG. 4). IFN-γ and NO responses to PPDb, however, were induced by M. avium and Map infection. Responses of these calves to PPDb exceeded (P<0.05) those of non-infected calves (FIGS. 4 and 5). NO responses of M. bovis-infected calves to PPDb exceeded (P<0.05) those of noninfected and M. avium-infected calves (FIG. 5). IFN-γ and NO responses of M. bovis-, M. avium-, and Map-infected calves to PPDa were comparable and exceeded (P<0.05) those of non-infected calves. IFN-γ and NO responses of M. bovis-infected calves to rESAT-6:CFP-10 exceeded those of noninfected, M. avium-, and Map-infected calves (FIGS. 4 and 5). IFN-γ and NO responses by M. avium- and Map-infected calves to rESAT-6:CFP-10, in contrast to stimulation with PPDb, did not exceed (P<0.05) those of non-infected calves. IFN-γ responses of M. bovis-infected calves to rESAT-6:CFP-10 were not different (P>0.05) at 4, 5, 6, and 7 months postinfection (FIG. 6).

### EXAMPLE 2

### Reindeer Study

Animals, M. bovis Culture, and Challenge Procedures.

Castrated male reindeer (Rangifer tarandus grantii, n=17) were obtained from a tuberculosis-free herd in Michigan and were housed at the National Animal Disease Center, USDA, ARS, Ames, Iowa according to institutional guidelines and approved animal care and use protocols. For M. bovis infection, the challenge inoculum [10⁵ colony forming units (cfu) in 0.2 ml of phosphate buffered saline, 0.15 M, pH 7.2 (PBS)] was instilled directly into the tonsilar crypts of anesthetized reindeer (n=13) as described for inoculation of white-tailed deer (Palmer M. V. et al., 1999, J. Wildl. Dis., 35:450-457). The strain of M. bovis used for the challenge inoculum (95-1315, USDA, Animal Plant and Health Inspection Service designation) was originally isolated from a white-tailed deer in Michigan, US. (Schmitt S. M. et al., 1997, J. Wildl. Dis., 33:749-58). Inoculum consisted of mid-log-phase M. bovis grown in Middlebrook's 7H9 media supplemented with 10% oleic acid-albumin-dextrose complex plus 0.05% Tween 80. At the time of inoculation, reindeer were moved from an outdoor pen into climate-controlled rooms (2-3 animals/room) within a biosafety level-3 (BL-3) confinement facility. All 4 non-infected reindeer were housed in a climate-controlled room in a separate building. At the initiation of the study, reindeer were approximately 9 months of age.

Whole Blood Cultures and Analysis of IFN-γ Production.

Heparinized blood samples were dispensed in 1.5 ml aliquots into individual wells of a 24-well plate. Treatments included no stimulation (PBS), M. avium PPD (Sigma Chemical Co.) (40 μg/ml), M. bovis PPD (Sigma Chemical Co.) (40 μg/ml), rESAT-6 (10 μg/ml), rCFP-10 (10 μg/ml), rESAT-6:CFP-10 (10 μg/ml), or pokeweed mitogen (PWM, Sigma Chemical Co.), 20 μg/ml. Cloning, expression, and purification of recombinant proteins was as described in Example 1. Blood cultures were incubated for 48 h, plasma harvested and stored at −80° C. IFN-γ concentrations in non-stimulated and stimulated plasma were determined using an ELISA-based kit (CSL Animal Health). Absorbencies of standards and test samples were read at 450 nm using an ELISA plate reader (Molecular Devices). Individual treatments were analyzed in duplicate. Data are presented as optical density readings (OD) or change in optical density readings upon stimulation as compared to no stimulation (i.e., ΔOD, antigen- or mitogen-stimulated sample OD minus non-stimulated sample OD).

Skin Testing Procedures.

Three months after M. bovis inoculation, reindeer were tested for in vivo responsiveness to mycobacterial antigens by a modified CCT technique enabling collection of biopsies of the dermal reactions to PPD's at 24-, 48-, and 72-hr post injection. Briefly the cervical region was clipped and animals injected intradermally in 3 separate locations with 0.1 ml M. bovis PPD (1 mg/ml) and a single location with 0.1 ml M. avium PPD (0.4 mg/ml). A standard CCT (i.e., single intradermal injection each of M. avium PPD and M. bovis PPD) was performed 8 months after M. bovis inoculation.

Statistics.

Data were analyzed by either Student's T test or one-way analysis of variance (ANOVA) followed by Tukey-Kramer multiple comparison's test using commercially available software (InStat 2.00, GraphPAD Software, San Diego, Calif.). Differences between groups were considered significant if probability values of P≦0.05 were obtained.

### Results

All M. bovis-inoculated reindeer had lesions consistent with M. bovis infection and infection was confirmed by isolation of M. bovis from tissues. Tuberculous lesions were detected within medial retropharyngeal lymph nodes from all infected reindeer. Lesions and M. bovis were also detected in tonsil, mesenteric lymph nodes, lung, and lung-associated lymph nodes. None of the reindeer had clinical signs of tuberculosis or disseminated disease even after 1 year of colonization, suggestive of low-grade chronic infection. M. bovis was not isolated from control reindeer.

Polyclonal stimulation of whole blood cultures with PWM resulted in significant (P<0.05) production of IFN-γ as compared to non-stimulated samples at all time points (data not shown). Responses to PWM were greatest from February to June and did not differ (P>0.05) between infected and non-infected reindeer. Mean responses to PWM stimulation over the course of the study (infected−1.03±0.06 ΔOD, n=201; non-infected−0.99±0.08 ΔOD, n=68) were similar to those presented for white-tailed deer (Palmer M. V. et al., 2004, J Vet Diagn Invest, 16:16-21). Mean responses to no stimulation (i.e., PBS) over the course of the study (infected−0.085±0.002 ΔOD, n=201; non-infected−0.086±0.003 ΔOD, n=68) were also similar to those presented for white-tailed deer (Palmer M. V. et al., 2004, J Vet Diagn Invest, 16:16-21). Infection status or time of year did not affect responses to no stimulation.

As early as 30d after inoculation and at most time points thereafter, responses by infected reindeer to M. bovis PPD exceeded (P<0.05) those by non-infected reindeer (data not shown). Responses to M. avium PPD did not differ between groups. Despite differences in responses to M. bovis PPD by the two groups, responses considered positive (i.e., >0.1 ΔOD) were detected by reindeer within the non-infected group. Each of the 4 non-infected reindeer had positive responses to M. bovis PPD, although not at each time point. Mean responses (i.e., average of individual responses over the course of the study, n=64) by non-infected reindeer to M. bovis PPD (0.47±0.06 ΔOD) exceeded (P<0.0001) concurrent responses to M. avium PPD (0.16±0.02 ΔOD).

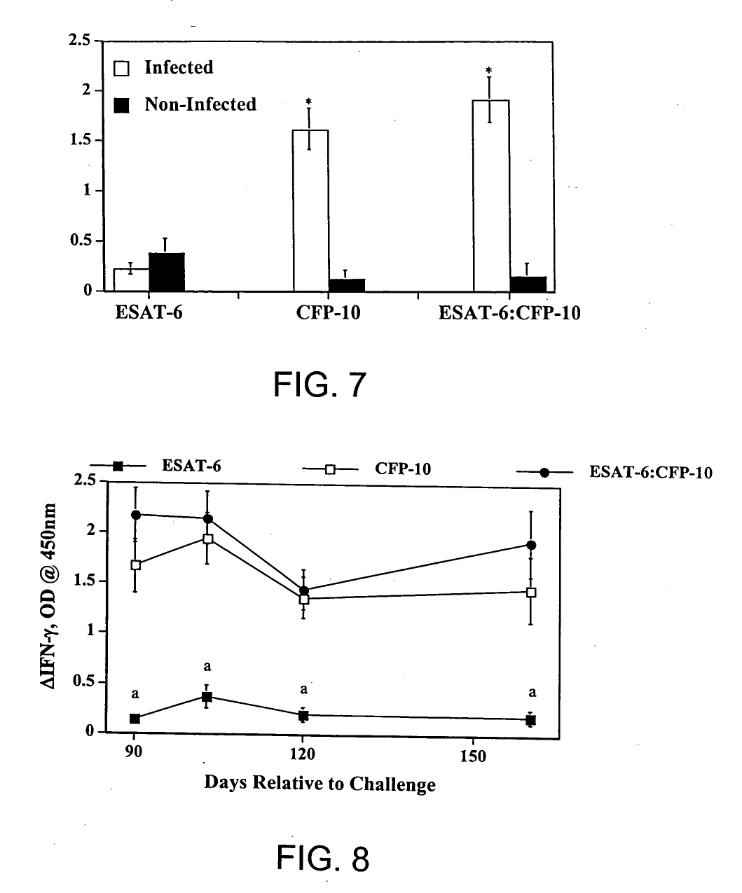

Antigens produced predominately by tuberculous mycobacteria and not by many environmental non-tuberculous mycobacteria were evaluated as an approach to increase the specificity of the test. Mean responses by infected reindeer to rCFP-10 and rESAT-6:CFP-10, but not rESAT-6, exceeded (P<0.01) respective responses by non-infected reindeer (FIG. 7). Responses by infected reindeer to rCFP-10 and rESAT-6:CFP-10 exceeded (P<0.01) responses to rESAT-6 at all time points tested (FIG. 8). Responses to rCFP-10 and rESAT-6:CFP-10, however, did not differ (P>0.05). Responses by infected reindeer to rCFP-10 (data not shown) and rESAT-6:CFP-10 (data not shown) exceeded (P<0.01) respective responses by non-infected reindeer at all time points. In contrast to M. bovis PPD, responses to rCFP-10 and rESAT-6:CFP-10 by ¾ non-infected reindeer were considered negative (i.e., <0.1 ΔOD) at each time point. One non-infected reindeer, however, had positive responses to rCFP-10 and rESAT-6:CFP-10 at each time point tested except one.

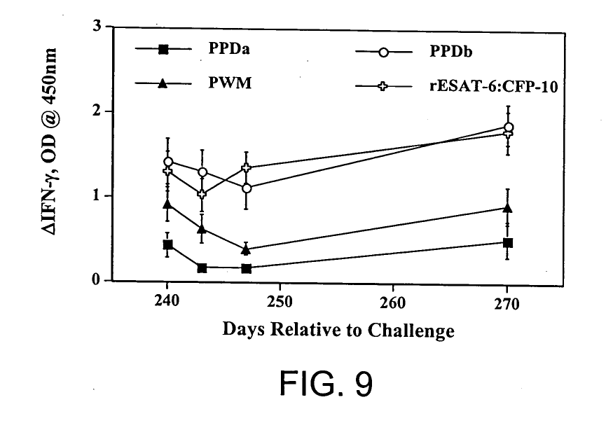

As skin testing is the only approved test for tuberculosis surveillance of reindeer within the US, it is likely that field evaluation of candidate tests will be initially performed in conjunction with skin testing. At 90 and 240 d after inoculation, experimentally infected reindeer were injected with PPD's for determination of in vivo responsiveness to mycobacterial antigens. With the initial test, animals received 3 injections of M. bovis PPD (300 μg) and 1 injection of M. avium PPD (40 μg). IFN-γ responses to M. avium PPD, M. bovis PPD, and PWM were reduced (P<0.05) 1 wk after PPD administration as compared to respective responses at the time of injection (data not shown). IFN-γ responses were only transiently depressed as responses returned to near pre-injection levels by 2 wks after PPD administration. For the second skin test at 8 months after inoculation, 100 μg M. bovis PPD and 40 μg M. avium PPD were injected following standard CCT technique. While not statistically significant, IFN-γ responses after this test were generally decreased for the 7 d period following injection (FIG. 9).

All references disclosed herein or relied upon in whole or in part in the description of the invention are incorporated by reference.

TABLE 1
Comparison of IFN-γ responses by PBMC from M. bovis-infected
cattle to responses by cattle naturally-sensitized to M. avium

| | Recall Antigen† | | |
|---|---|---|---|
| Group | PPDa | PPDb | rESAT-6: CFP-10 |
| M. bovis-infected | 17.3* (3.0) | 55.2** (9.7) | 29.1** (6.1) |
| (6 months postinfection) | | | |
| M. avium-exposed | 7.5 (3.0) | −2.4 (1.1) | −2.9 (0.7) |
| (Natural Exposure) | | | |

†Values represent mean (±SEM, n = 8) IFN-γ responses (ng/ml) of PBMC to antigen stimulation minus their response to media alone after 48 h. For a specific stimulation, asterisks indicate that responses of cattle infected with M. bovis differ from the corresponding response by M. avium-exposed cattle (*P < 0.05, **P < 0.01).

TABLE 2
Comparison of TFN-α responses by PBMC from M. bovis-infected
cattle to responses by cattle naturally-sensitized to M. avium

| | Recall Antigen† | | |
|---|---|---|---|
| Group | PPDa | PPDb | rESAT-6: CFP-10 |
| M. bovis-infected | 0.316 (.084) | 1.370* (0.524) | 0.790* (0.285) |
| (6 months | | | |
| postinfection) | | | |
| M. avium-exposed | 0.164 (0.048) | 0.056 (0.052) | 0.038 (0.095) |
| (Natural Exposure) | | | |

†Values represent mean (±SEM, n = 8) TFN-α responses (ng/ml) of PBMC to antigen stimulation minus their response to media alone after 48 h. For a specific stimulation, asterisks indicate that responses of cattle infected with M. bovis differ from the corresponding response by M. avium-exposed cattle (*P < 0.05).

TABLE 3
Comparison of NO responses by PBMC from M. bovis-infected
cattle to responses by cattle naturally-sensitized to M. avium

| | Recall Antigen† | | |
|---|---|---|---|
| Group | PPDa | PPDb | rESAT-6: CFP-10 |
| M. bovis-infected | 196.1** (46.1) | 403.3** (86.4) | 415.7** (98.4) |
| (6 months | | | |
| postinfection) | | | |
| M. avium-exposed | 49.1 (9.1) | −4.5 (9.9) | −19.0 (10.1) |
| (Natural Exposure) | | | |

†Values represent mean (±SEM, n = 8) NO responses (ng/ml) of PBMC to antigen stimulation minus their response to media alone after 72 h. For a specific stimulation, asterisks indicate that responses of cattle infected with M. bovis differ from the corresponding response by M. avium-exposed cattle (**P < 0.01).

TABLE 4
IFN-γ responses to crude mycobacterial antigens
upon experimental infection

| | Recall Antigen (whole cell sonicates)† | | |
|---|---|---|---|
| | M. bovis | M. avium | |
| Infection Status | strain 1315 | strain 3988 | Map strain K10 |
| Non-infected | −0.7 (0.4)a | −1.0 (0.4)a | −0.8 (0.4)a |
| M. bovis-infected | 60.0* (2.5)c | 15.2 (0.9)b | 9.2 (0.7)b |
| M. avium-infected | 13.1 (2.4)b | 29.6* (2.9)c | 16.8 (2.5)c |
| Map-infected | 17.0 (5.2)b | 22.7 (7.5)b, c | 30.9 (11.6)b, c |
| †IFN-γ responses (whole blood assay) were unaffected (P > 0.05) by length of postinfection period. For this reason, values represent mean (±SEM, ng/ml) IFN-γ responses (i.e., response to 20 g/ml antigen minus response to media alone after 48 h) during the period spanning 4 to 7 months postinfection (n = 4). | | | |
| # Treatment groups consisted of 4 animals except for the Map-infected group that had 3 animals. | | | |

*Asterisks indicate treatment means for a specific type of infection (i.e., horizontal comparisons) differ (P < 0.05).
a-cLetters indicate treatment means for a specific type of stimulation (i.e., vertical comparisons) differ (P < 0.05).

TABLE 5
Nitric oxide responses to crude mycobacterial antigens
upon experimental infection

| | Recall Antigen (whole cell sonicates)† | | |
|---|---|---|---|
| | M. bovis | M. avium | |
| Infection Status | strain 1315 | strain 3988 | Map strain K10 |
| Non-infected | −106 (53) a | −49 (51) a | −63 (43) a |
| M. bovis-infected | 451* (46) c | 184 (21) b | 97 (35) a, b |
| M. avium-infected | 79 (17) b | 185* (20) b | 55 (14) a, b |
| Map-infected | 87 (52) a, b | 213 (61) b | 278 (117) b |
| †NO responses by PBMC were unaffected (P > 0.05) by length of postinfection period. For this reason, values represent mean (±SEM, ng/ml) NO responses (i.e., response to 20 g/ml antigen minus response to media alone after 72 h) during the period spanning 5 to 7 months postinfection (n = 3). Treatment groups | | | |
| # consisted of 4 animals except for the Map-infected group that had 3 animals. | | | |

*Asterisks indicate treatment means for a specific type of infection (i.e., horizontal comparisons) differ (P < 0.05).
a-c Letters indicate treatment means for a specific type of stimulation (i.e., vertical comparisons) differ (P < 0.05).

We claim:

1. A fusion protein comprising ESAT-6 and CFP-10 or a variation thereof that stimulates a T-cell response to Mycobacterium bovis that is significantly greater than a T-cell response to a non-tuberculous mycobacterium in the absence of stimulation by said fusion protein.

2. The fusion protein of claim 1, wherein said T-cell response is induction of cytokine secretion.

3. The fusion protein of claim 1, wherein said T-cell response is induction of IFN-γ secretion.

4. The fusion protein of claim 1, wherein said ESAT-6 is characterized by SEQ ID NO:4.

5. The fusion protein of claim 1, wherein said CFP-10 is characterized by SEQ ID NO:6.

6. The fusion protein of claim 1, wherein said variation is one or more of a deletion, addition, or substitution in ESAT-6, CFP-10 or both ESAT-6 and CFP-10.

7. The fusion protein of claim 1, further comprising one or more restriction enzyme sites.

8. The fusion protein of claim 1, further comprising one or more linkers.

9. The fusion protein of claim 1 comprising the sequence of SEQ ID NO:2.

10. The fusion protein of claim 1 encoded by SEQ ID NO:1.

11. The fusion protein of claim 1 encoded by a sequence comprising SEQ ID NOs:3 and 5.

12. The fusion protein of claim 1 comprising SEQ ID NOs:4 and 6.

13. A method for differentiating infection of an animal with Mycobacterium bovis from exposure of said animal to other species of Mycobacteria comprising:
a. stimulating a cell of said animal with the fusion protein of claim 1 to produce an immunogenic response;
b. measuring said response of said cell to the stimulation in step (a).
c. comparing said response in (b) to a predetermined scale of responses indicative of said infection.

14. The method of claim 13, wherein said stimulating a cell is in vivo.

15. The method of claim 14, wherein said response is a hypersensitivity skin response.

16. The method of claim 13, wherein said stimulating a cell is in vitro.

17. The method of claim 16, wherein said cell is a blood cell.

18. The method of claim 13, wherein said response is an IFN-γ response.

19. The method of claim 13, wherein said response is a nitric oxide response.

20. The method of claim 13, wherein said response is a TNF-α response.

21. The method of claim 13, wherein said other species of Mycobacteria is selected from the group consisting of M. avium and M. avium subspecies paratuberculosis.
